(19) United States
(12) Patent Application Publication
(10) Pub. No.: US 2009/0132030 A1
(43) Pub. Date: May 21, 2009

(54) Method Of Modifying A Metal Substrate To Improve Surface Coverage Of A Coating

(75) Inventors: Mao-Jung Maurice Lien, Maple Ridge (CA); Doug Smith, Vancouver (CA); Arc Rajtar, Port Moody (CA)

Correspondence Address: RISSMAN JOBSE HENDRICKS & OLIVERIO, LLP, 100 Cambridge Street, Suite 2101, BOSTON, MA, 02114, US

(73) Assignee: MIV Therapeutics Inc., Vancouver, BC (CA)

(21) Appl. No.: 11/574,440

(22) PCT Filed: Aug. 30, 2004
(86) PCT No.: PCT/CA04/01585

§ 371 Date: Sep. 26, 2008

(51) Int. Cl. A61F 2/06; B05D 3/02; B32B 15/00; B32B 15/04

(52) U.S. Cl. 623/1.42; 427/318; 427/248.1; 427/2.25; 427/2.24; 428/457

(57) ABSTRACT

This application relates to a method of modifying the surface of a metal substrate to improve the surface coverage of a coating applied to the substrate. The method comprises heating at least the surface of the substrate to a temperature within the range of approximately 175-400° C.; and applying at least one layer of the coating to the substrate. In one particular embodiment the substrate is heated to a temperature within the range of 200-350° C. The low temperature heating enhances the hydrophilicity of the metal substrate while avoiding the disadvantages of high temperature thermal oxidation.

### TECHNICAL FIELD

This application relates to a method of modifying the surface of a metal substrate to improve the surface coverage of a coating applied to the substrate.

### BACKGROUND

Metallic biomaterials are used in medical devices due to their superior strength, biocompatability, durability and resistance to corrosion in physiological environments. In the case of implantable medical devices such as stents, stainless steel or cobalt-chromium steel metallic substrates are commonly coated with a thin layer of a ceramic, such as hydroxyapatite. Hydroxyapatite is chemically similar to the mineral component of bones and hard tissue in mammals and is one of the few materials that is classified as bioactive and fully biocompatible. Hydroxyapatite may be coated on a metal substrate using sol-gel deposition techniques employing, for example, aerosols, spin-coating or dip-coating. It is often very difficult to achieve even coverage of ultra-thin coatings, particularly in the case of medical devices such as stents having complicated three-dimensional geometries.

Methodologies for modifying the surface of implantable medical devices and the like to facilitate deposition of thin film coatings are known in the prior art. U.S. Pat. No. 4,818,572, Shimamune et al., describes a process for producing a calcium phosphate-coated composite material which comprises oxidizing a metallic substrate to form a layer of an oxide of the metal component of the substrate on the surface of the substrate, and forming a coating layer of a calcium phosphate compound on the surface of the oxide layer. The substrate metals may include stainless steel and cobalt-chromium base alloys. The thermal oxidation process outlined in the Shimamune et al. disclosure uses temperatures in the range of 400 to 1000° C. Shimamune et al. also describe electrolytic oxidation processes.

While Shimamune et al. does suggest that thermal oxidation pre-treatment can improve the adhesion and uniformity of a subsequently deposited coating layer, the oxidation is performed at relatively high temperatures (i.e. above 400° C.). There are numerous disadvantages to high temperature oxidation of metal substrate surfaces. For example, high temperature oxidation of nickel alloys, such as stainless steel and cobalt-chromium alloys, can result in surface layers enriched in elemental nickel.¹ These nickel enriched surface sub-layers are a source of nickel that can be potentially released in the body environment. In the case of implantable medical devices such as stents, in vivo release of nickel and chromium may potentially cause deleterious side effects. For example, leaching of nickel from implanted stents may cause chronic inflammation and has been associated with increased risk of in-stent restenosis.² Other studies also suggest that stainless steel coronary stents may trigger allergic reactions to such metals as molybdenum, chromium and nickel.^{3, 4} ¹ Svetlana A. Shabalovskaya, Surface, corrosion and biocompatibility aspects of Nitinol as an implant material, Bio-Medical Materials and Engineering 12 (2002) 69-109 at 73.² McClean et al., Stent Design: Implications for Restenosis, Reviews in Cardiovascular Medicine, Vol. 3, Suppl. 5 2002, S16-S22 at p. S18.³ Koster et al., Nickel and molybdenum contact allergies in patients with coronary in-stent restenosis, Lancet 2000; 356:1895-1897⁴ Assad et al., Porous Titanium-Nickel for Intervertebral Fusion in a Sheep Model: Part 2. Surface Analysis and Nickel Release Assessment. J Biomed Mater Res Part B: Appl Biomater 64B: 121-129, 2003.

Further, high temperature treatment can change the mechanical properties of metal substrates. If metal stents are subjected to temperatures above a stress-relieving temperature, then the mechanical properties of the metal may be compromised to the extent that the stents are unsuitable for in vivo implantatation. Metal recoil and fatigue characteristics are important factors in stent design.³

Moreover, stent surface characteristics may be relevant to risk of thrombois and restenosis. For example, microscopic roughness caused by thermal oxidation and the like may increase platelet adhesion in vivo which is associated with thrombogenicity. Also, for aesthetic and marketing reasons it is desirable that stents and other medical devices have an ultra-smooth, uniform appearance and hence excessive thermal oxidation should be avoided.

The need has therefore arisen for a method of improving the surface coverage of coatings applied to metal substrates utilizing low temperature thermal pre-treatment prior to the coating step(s).

### SUMMARY OF INVENTION

In accordance with the invention, a method of modifying the surface of a metal substrate to improve the surface coverage of a coating applied to the substrate is provided. The method comprises heating at least the surface of the substrate to a temperature within the range of approximately 175-400° C.; and applying at least one layer of the coating to the substrate. In one particular embodiment the substrate is heated to a temperature within the range of 200-350° C. The low temperature heating enhances the hydrophilicity of the metal substrate, and therefore increases the coverage of hydrophobic coatings, while avoiding the disadvantages of high temperature thermal oxidation.

The substrate may comprise steel or a steel alloy. For example, the substrate may be selected from the group consisting of stainless steel and cobalt chromium steel. In one embodiment the substrate may be an implantable medical device, such as a stent.

The coating may be applied to the substrate surface as an aerosol. In one embodiment the coating is applied in a sol-gel process. The coating may comprise calcium phosphate compound and/or a ceramic compound such as hydoxyapatite. The coating may be applied in droplets, for example using an aerosol nebulizer. The method reduces the surface tension of the substrate and improves the coverage of the coating such that the contact angle of the droplets is less than 10°, and preferably less than 5°.

The coating is preferably applied to the substrate less than 24 hours following the heating step. In one embodiment the coating is applied immediately following the heating step. In another embodiment at least some of the coating may applied to the substrate prior to or simultaneous with the heating step. Multiple layers of the coating may optionally be applied to the substrate. The method may further include the step of sintering the coated substrate at a temperature exceeding 400° C.

The invention also pertains to a coated substrate produced by the method described herein, such as a coated substrate configured for use as a stent or other implantable medical device.

### BRIEF DESCRIPTION OF DRAWINGS

In drawings which illustrate embodiments of the invention, but which should not be construed as restricting the spirit or scope of the invention in any way,

FIGS. 1(a) and 1(b) are photographs showing the surface morphology of electropolished stent substrates: (a) SS31L stent and (b) Co—Cr stent.

FIGS. 2(a)-2(f) are graphs showing oxidation weight changes on a stainless steel substrate as a function of heating temperature and heating time. The thermal gravimetric heating profiles are at: (a) 150° C.; (b) 200° C.; (c) 350° C.; (d) 375° C.; (e) 450° C.; and (f) 550° C.

FIGS. 3(a)-3(e) are photographs showing the contact angle of hydroxyapatite sol droplets deposited on polished stainless steel plates as function of heat treatment temperature: (a) no heat treatment; (b) heat treated at 150° C.; (c) heat treated at 200° C.; (d) heat treated at 350° C.; (e) heat treated at 450° C; (f) heat treated at 550° C.

FIGS. 4(a) and 4(b) are photographs taken with an optical microscope showing SEM surface morphology of hydroxyapatite coatings applied without thermal pre-treatment on: (a) a SS316L stent substrate; and (b) Co—Cr stent substrate. The substrates were coated in an aero-sol process and fired at 500° C.

FIGS. 5(a) and 5(b) are additional photographs taken with an optical microscope showing SEM surface morphology of hydroxyapatite coatings applied without thermal pre-treatment on: (a) a SS316L stent substrate; and (b) Co—Cr stent substrate. The substrates were coated in a process similar to FIGS. 4(a) and 4(b).

FIGS. 6(a)-6(f) are are photographs taken with an optical microscope showing scanning electron microscopy (SEM) surface morphology of hydroxyapatite coatings applied to a SS316L stent substrate after thermal pre-treatment at various temperatures: (a) 150° C.; (b) 200° C.; (c) 350° C.; (d) 375° C.; (e) 450° C.; and (f) 550° C.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

The present invention relates to surface modification of metal substrates to improve surface coverage of coatings applied to the substrates. Although the invention is described herein in relation to implantable medical devices such as stents, the invention may be useful in any application where improved coating coverage is desirable.

In one application of the invention the metal substrates may comprise stainless steel or cobalt chromium steel alloys and the coating may be a ceramic, such as hydroxyapatite, deposited as a thin film. Other coatings suitable for deposition on metal surfaces may also be employed. As will be familiar to a person skilled in the art, various sol-gel deposition techniques are known for achieving thin film coatings. Such sol-gel techniques may include spin-coating, dip-coating and aerosol processes. Aerosol techniques may employ an ultrasonic nebulizer for applying sol in droplet form. However, it is often difficult to achieve even coverage. In many cases high substrate surface tension may result in droplet patches and lack of uniform coating coverage.

The present invention improves coating coverage by means of thermal pre-treatment. The inventors have determined that markedly improved coating coverage may be achieved using relatively low pre-treatment temperatures (i.e. less than 400° C.). Low temperature pre-treatment has numerous advantages as described herein. For example, low temperature pre-treatment avoids excessive thermal oxidation of the substrate which could otherwise result in exposure of potentially hazardous metal elements, such as nickel and chromium. Moreover, low temperature pre-treatment avoids subjecting stents or other metal substrates to stress-relieving temperatures. Such high temperatures could alter a substrate's mechanical properties and make it unsuitable for implantation in vivo. Apart from safety concerns, low temperature pre-treatment is also faster and more cost-effective than high temperature techniques.

The exact mechanism by which low temperature pre-treatment alters the surface morphology of the metallic substrate is the subject of on-going study. It is believed that partial thermal oxidation of the substrate modifies the outer surface layer to reduce its surface tension and enhance its hydrophilicity.

As will be appreciated by a person skilled in the art, many variations in the thermal pre-treatment and coating processes are possible without departing from the invention. For example, the benefits of low temperature thermal pre-treatment appear to be time limited. The coating is therefore preferably applied to the substrate less than 24 hours following the heating step. In one embodiment the coating is applied immediately following the heating step. In another embodiment at least some of the coating may applied to the substrate prior to or simultaneous with the heating step. Multiple layers of the coating may optionally be applied to the substrate. The method may further include the step of sintering the coated substrate at a high temperature (i.e. exceeding 400° C.) after the coating process is complete.

The following examples will illustrate the invention in further detail although it is appreciated the invention is not limited to the specific examples.

### EXAMPLES

### Experimental Methods

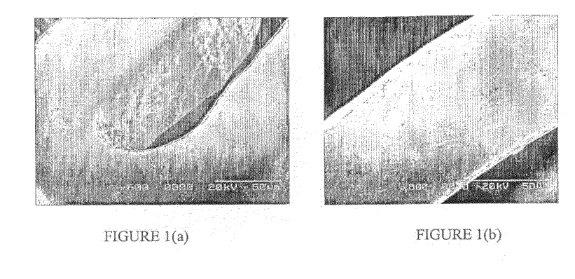

A thin film precursor sol was prepared first hydrolyzing triethyl phosphate (Fisher, USA) for 24 hours with a fixed amount of distilled water in a paraffin sealed glass container under vigorous stirring. A stoichiometric amount of 4M aqueous calcium nitrate (Aldrich, USA) solution was added dropwise into the hydrolyzed phosphite sol. The mixed sol solution was then continuously agitated for additional 3 minutes and kept static at ambient temperature for 24 hours. Stainless steel (SS316L) and cobalt chromium (Co—Cr) steel substrates were electro-polished for the purpose of maintaining constant surface finish with the resulting surface morphology (˜100 nm roughness) shown in FIG. 1. These substrates were subsequently heat treated at 150° C., 200° C., 350° C., 375° C., 450° C. and 550° C. temperatures for 10 minutes respectively. Thermal Gravimetric Analysis (TGA) was used to monitor correlation between weight changes, the temperature and the time during pre-heating process on all substrates. Contact angles of hydroxyapatite (HAp) drops on preheated stainless steel plate substrates were also measured to assist in evaluation of low temperature oxidation impact at the surface tension on SS316L and cobalt chromium substrates. Subsequently hydroxyapatite thin film was deposited on these pretreated substrates using aerosol nebulizing process for 40, 60 and 120 seconds respectively. The coatings were then sintered at 500° C. for 40 minutes. The coated substrates were examined under scanning electron microscopy (SEM) and energy disperse X-ray (EDX) operated at 10 kV to evaluate the HAp coating coverage and quantify the coating composition as discussed below.

### Thermal Gravimetric Analysis

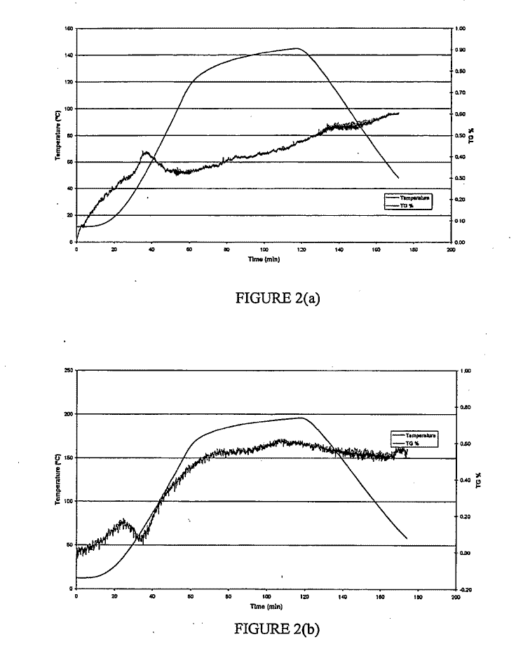

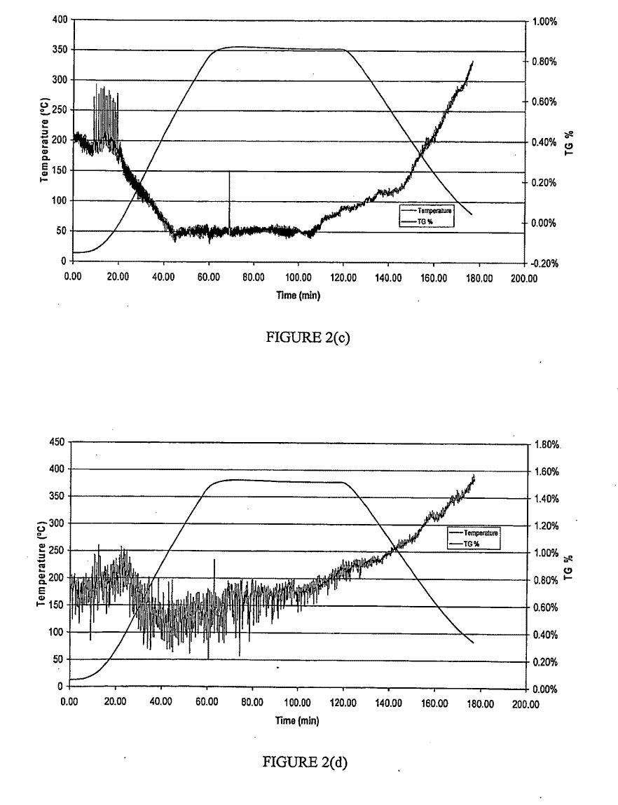

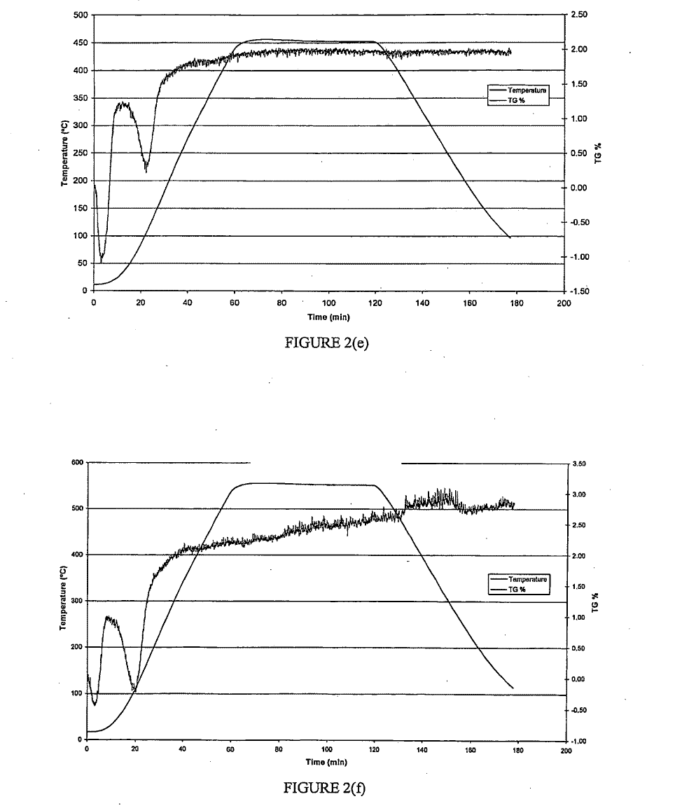

As shown in FIGS. 2(a)-2(f), thermal gravimetric analysis (TGA) results demonstrated that oxidation weight changes on stainless steel plate substrate were directly related to heating temperature and the heating time. Higher heating temperature increased oxidation amount reflected through higher weight gains. The results indicate that oxidation will reach a saturation point (i.e. when the oxidation process is finished). The results also indicate that low temperature oxidation does occur. Samples processed at lower temperatures, (i.e. 150° C. and 200° C.) exhibited continuous weight change and did not reach a saturation point (FIGS. 2(a) and 2(b)).

### Contact Angle Investigation

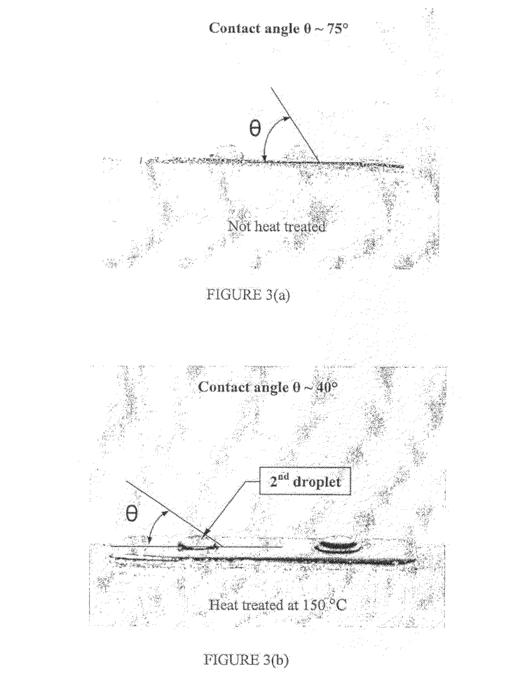

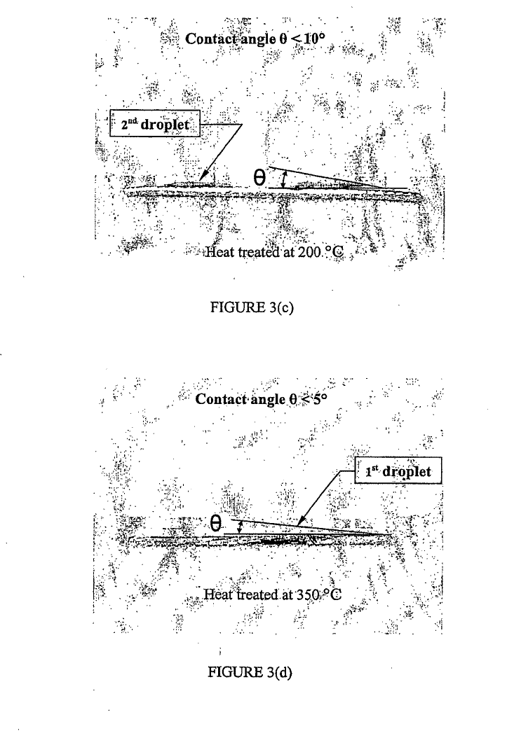

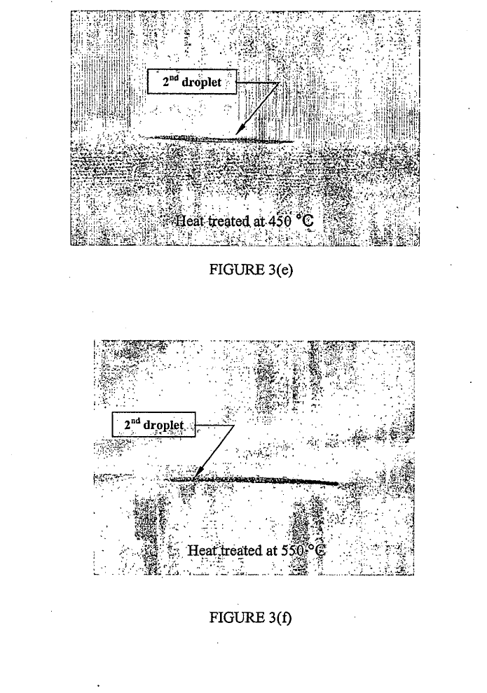

Polished stainless steel plates with HAp sol droplets deposited thereon were investigated to determine changes in droplet contact angle resulting from heat treatment at different temperatures. As shown in FIGS. 3(a)-3(e), one untreated plate and five heat treated plates were considered. Without heat treatment (FIG. 3(a)), the contact angle between the droplet and polished substrate was relatively high (i.e. greater than 75°). This result indicates the untreated sample exhibited poor wetability and hydrophilicity. The same samples demonstrated tremendous improvement of contact angle following heat treatment at temperatures above 150° C. (FIG. 3(b)-3(e)). For example, in the sample subjected to relatively modes pre-treatment at 200° C., the contact angle was reduced to less than 10°.

### Scanning Electron Microscopy

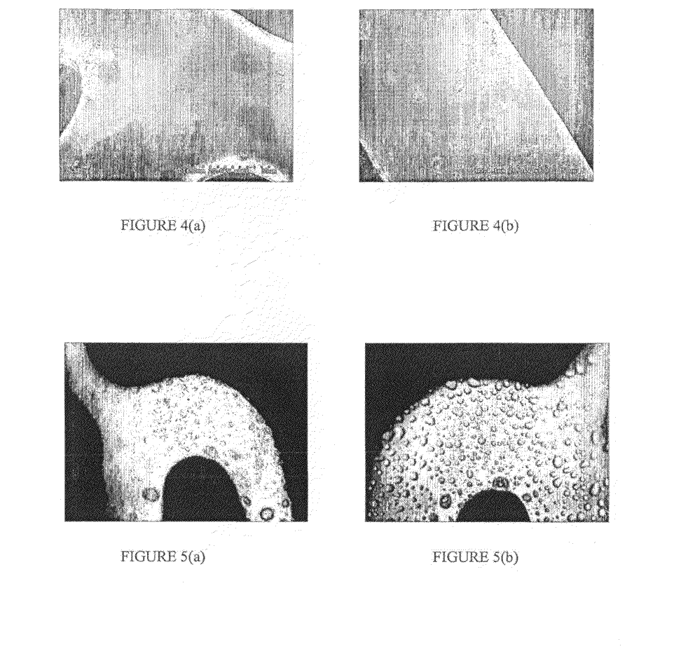

Surface morphology of HAp coating on both SS316L (FIG. 4(a) and Co—Cr (FIG. 4(b) stent substrates was also investigated by scanning electron microscopy. All substrates were coated in an aero-sol process and fired at 500° C. The coatings clearly exhibited droplet-like patches attributed to poor hydrophilic properties of the substrate surface.

Similarly, FIGS. 5(a) and 5(b) show the surface microstructure of the HAp coating on both SS316L and Co—Cr stent substrates without heating pre-treatment. The coating exhibited a patch pattern with coverage less than 40% of the coated surface.

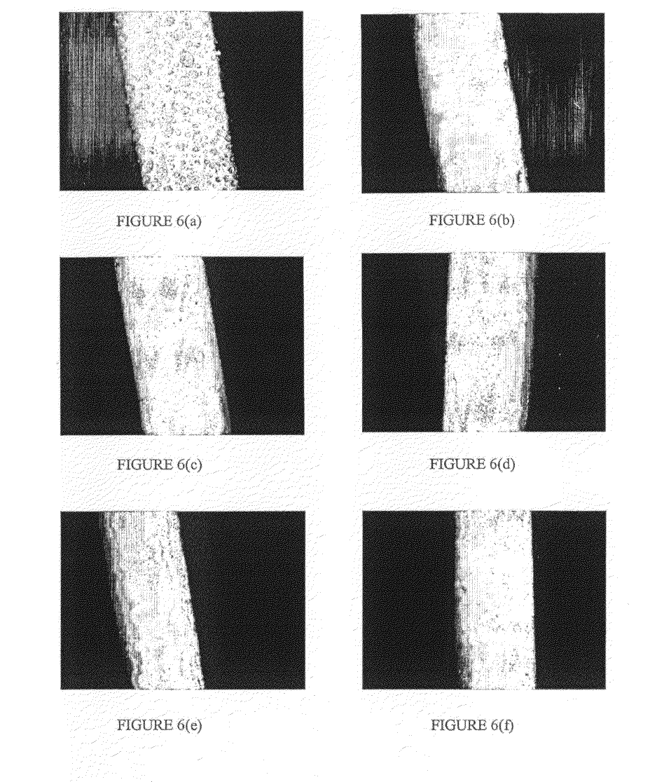

FIGS. 6(a)-6(f) shows surface microstructure of HAp coatings on pre-heated SS316L stent substrates. The substrates were pre-heated at different temperatures for 40 minutes, aero-sol coated and fired at 500° C. The sample pre-heated at 150° C. (FIG. 6(a)) did not demonstrate improvement of coating surface coverage. However, the sample pre-heated at 200° C. (FIG. 6(b)) did show a significant improvement in coating coverage. In particular, this sample exhibited coverage exceeding approximately 80% of the coated surface. Pre-treatment at higher temperatures (FIG. 6(c)-(f)) showed similar improvements in coating coverage. However, the results at higher pre-treatment temperatures were not dramatically superior to the 200° C. sample. This suggests that heating pre-treatment of SS316L and Co—Cr alloy substrates at comparatively low temperatures (i.e. above approximately 175-200° C.) can modify the morphology of substrate surface to enhance coverage of a subsequently applied coating. The heating pre-treatment appears to modify the morphology of the substrate to improves its hydrophilicity or “wetabiliy” and reduce surface tension.

### Energy Disperse X-ray Analysis

Table 1 below shows the results of surface composition analysis of a HAp thin film coating deposited on a SS316L stent substrate by a aero-sol coating process following low temperature pre-treatment. Energy Disperse X-ray (EDX) was used to analyze the presence of calcium and phosphorus. This analysis demonstrated that the HAp thin film coating was widely present and homogenous on the substrate surface.

TABLE 1

| Composition | Concentration (wt %) |
|---|---|
| Carbon | 5.55 |
| Oxygen | 9.30 |
| Silicon | 0.29 |
| Phosphorous | 2.08 |
| Sulfur | 1.08 |
| Calcium | 2.84 |
| Chromium | 14.86 |
| Manganese | 1.42 |
| Iron | 50.93 |
| Nickel | 11.65 |

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

What is claimed is:

1. A method of modifying the surface of a metal substrate to improve the surface coverage of a coating applied to said substrate comprising:
(a) heating at least said surface of said substrate to a temperature within the range of approximately 175-300° C.; and
(b) after said heating applying at least one layer of said coating to said substrate.

2. The method as defined in claim 1, wherein said heating enhances the hydrophilicity of said metal substrate.

3. The method as defined in claim 1, wherein said coating is hydrophobic.

4. The method as defined in claim 1, wherein said substrate comprises steel or a steel alloy.

5. The method as defined in claim 4, wherein said substrate is selected from the group consisting of stainless steel and cobalt chromium steel.

6. The method as defined in claim 1, wherein said coating is applied in a sol-gel process.

7. The method as defined in claim 6, wherein said coating is applied by a technique selected from the group consisting of aerosol deposition, spin-coating, dip-coating and vapor deposition.

8. The method as defined in claim 1, wherein said coating is a calcium phosphate compound.

9. The method as defined in claim 1, wherein said coating is a ceramic.

10. The method as defined in claim 1, wherein said coating is hydoxyapatite.

11. The method as defined in claim 1, wherein said surface of said substrate is heated to a temperature within the range of 200-300° C.

12. The method as defined in claim 1, wherein said coating is applied to said substrate less than 24 hours following said heating step.

13. The method as defined in claim 12, wherein said coating is applied to said substrate immediately following said heating step.

14. The method as defined in claim 1, wherein multiple layers of said coating are applied to said substrate.

15. The method as defined in claim 1, wherein said coating is applied as droplets using an aerosol nebulizer and wherein the contact angle of said droplets is less than 10°.

16. The method as defined in claim 15, wherein said contact angle of said droplets is less than 5°.

17. The method as defined in claim 4, wherein said substrate is an implantable medical device.

18. The method as defined in claim 17, wherein said implantable medical device is a stent.

19. The method as defined in claim 18, further comprising sintering said substrate at a temperature exceeding 400° C.

20. The method as defined in claim 1, wherein said heating causes thermal oxidation of said metal substrate.

21. The method as defined in claim 1, wherein said heating is performed in an inert atmosphere.

22. The method as defined in claim 1, wherein at least some of said coating is applied to said substrate prior to or simultaneous with said heating step.

23. The method as defined in claim 1, wherein said heating reduces the surface tension of said metal.

24. A coated substrate produced by the method of claim 1.

25. The coated substrate of claim 21 configured for use as a stent.

26. A method of modifying the surface of a metal substrate to improve the surface coverage of a coating applied to said substrate comprising:
(a) heating at least said surface of said substrate to a temperature within the range of approximately 175-300° C.; and
(b) after the step of paragraph (a), applying at least one layer of said coating to said substrate,
wherein said substrate is selected from the group consisting of stainless steel and cobalt chromium steel and wherein said coating is hydoxyapatite.

27. The method as defined in claim 1, wherein said substrate is uncoated prior to said heating step.

28. The method as defined in claim 1, wherein said heating occurs in the absence of surface modifying or electrolytic agents.

29. A method of modifying the surface of metal substrate to improve the surface coverage of a coating applied to said substrate comprising:
(a) heating at least said surface of said substrate to a temperature within the range of approximately 175-300° C.; and
(b) applying at least one layer of said coating to said substrate,
wherein at least some of said coating is applied to said substrate prior to or simultaneously with said heating step.
